(19) United States
(12) Patent Application Publication
(10) Pub. No.: US 2005/0084461 A1
(43) Pub. Date: Apr. 21, 2005

(54) Tooth enamel rejuvenating toothpaste

(76) Inventors: Anthony E. Winston, East Brunswick, NJ (US); Bruce J. Nelson, Dayton, NJ (US)

Correspondence Address: Stephen B. Shear;Church & Dwight Co., Inc., 469 North Harrison Street, Princeton, NJ, 08543, US

(21) Appl. No.: 10/686,911

(22) Filed: Oct. 16, 2003

(51) Int. Cl. A61K 7/16; A61K 7/18

(52) U.S. Cl. 424/52

(57) ABSTRACT

An oral composition is provided which is effective for mineralization of surface enamel comprising a first part containing a partially water soluble calcium salt and a second part containing a fluoride salt and a bicarbonate salt. The oral composition can be a toothpaste and wherein the second part preferably contains sufficient bicarbonate salt to provide a fresh, clean feeling to the oral cavity.

### BACKGROUND OF THE INVENTION

This invention relates to oral products. More particularly, this invention relates to calcium, fluoride, and bicarbonate-containing toothpaste products capable of mineralizing the surface of tooth enamel.

Tooth enamel, the hard outer portion of teeth, is composed of 3-5μ diameter horseshoe-shaped rods surrounded by interstitial material. The rods primarily consist of closely packed columns of inorganic carbonated calcium hydroxyapatite crystals. The interstitial material is also mostly inorganic crystals of carbonated calcium hydroxyapatite. Normally, at the surface of tooth enamel there is a smooth layer of mineral, which partially or completely covers the rods. However, various imperfections are often present in the surface enamel layer. For example, one can sometimes see somewhat regularly-spaced dimples and ripples where the enamel rods appear to partially break through the surface.

Imperfections can also be present in tooth enamel due to exposure of teeth to various kinds of challenges and traumas. Thus, for example, many foods and beverages contain acids, which can erode the enamel surface of teeth, exposing the enamel rods, roughening the surface, and reducing surface gloss.

The surface of tooth enamel can also become damaged and scratched due to contact with hard instruments or even hard particles of foods. Alternatively, the surface of enamel can be damaged by highly abrasive prophy pastes applied by a dentist or by the use of highly abrasive toothpaste.

It should be noted that the cause of enamel surface imperfections is different than the caries disease process, which is caused by acidogenic bacteria. The caries process causes the formation of subsurface demineralized lesions within the enamel but initially leaves an intact enamel surface. Sometimes, these lesions are visible as “white spots” on the surface of the enamel. If the caries process is unchecked, enamel within and above a subsurface lesion eventually collapses, leading to cavitation and subsequent loss of tooth structure.

Remineralization is a natural process whereby calcium and phosphate from saliva reforms lost mineral in lesions in teeth. Remineralization is enhanced in the presence of fluoride ions. Therefore, fluoride is usually added to dentifrices as an active anti-caries ingredient which promotes the remineralization of teeth. However, the ability of fluoride to promote remineralization can be limited by the availability of calcium and phosphate ions in saliva. Therefore, oral products utilizing calcium and phosphate ions have been designed to enhance fluoride's ability to remineralize lesions and are disclosed, for example, in U.S. Pat. No. 4,080,440 (DiGiulio et al.); U.S. Pat. Nos. 4,177,258; 4,183,915; and 4,348,381 (all to Gaffar et al.); U.S. Pat. Nos. 4,606,912 and 4,610,873 (both to Rudy et al.); U.S. Pat. Nos. 5,037,639; 5,268,167; 5,437,857; 5,427,768 and 5,460,803 (all to Tung); U.S. Pat. No. 5,605,677 (Schumann et al.); U.S. Pat. Nos. 5,603,922; 5,605,675; 6,149,448; 5,833,957 and 5,858,333 (all to Winston and Usen).

In the remineralizing/mineralizing oral products disclosed in the above-cited references, remineralization and tubule mineralization are achieved by providing fluoride supplemented with calcium and phosphate ions in the oral cavity. However, the addition of calcium, phosphate, and fluoride ions to the oral cavity in an effective form and by application of dentifrices is not a simple matter. Calcium ions are rather reactive with phosphate and fluoride ingredients. Thus, for example, calcium cannot just be added to dentifrices in the presence of phosphate ions because the two will react with each other in the product to form an insoluble calcium phosphate, which is not effective for remineralization. By the same token, in a fluoride dentifrice, calcium ions would react readily with fluoride ions to precipitate insoluble calcium fluoride. The consequent reduction of soluble fluoride would reduce or eliminate the anti-caries activity thereof, and the remineralizing effects of the ingredients would be lost.

In the oral products disclosed in the prior art references cited above, premature reactions between the calcium, phosphate, and fluoride salts are avoided by keeping the calcium salt physically separate from the phosphate and fluoride salts. In one type of method, the calcium and phosphate salts are applied sequentially onto tooth surfaces. This method of application of a dentifrice does not present a commercially viable oral care product. Alternatively, disposing either or both of the cationic (calcium) and anionic (phosphate/fluoride) parts in a non-aqueous medium, or adding a stabilizing agent (e.g., chelating agent, antinucleation agent) to the product at a low pH, e.g., less than 4.0, are also disclosed as remineralizing products. The formulation of toothpastes in non-aqueous media can result in difficulties including undesirable slow release of the fluoride, calcium and phosphate ions. Also, among other problems, the formulation of toothpastes with highly acidic pH levels can present an irritation risk and can result in an unpleasant tasting product.

It is believed that the first enhanced remineralizing dentifrice made available to the public was based on the Winston and Usen patents mentioned previously. These patents describe a two-part product comprising a first part comprising a calcium salt and a second part comprising phosphate and fluoride salts which are kept separate in a tube by a physical divider but wherein both parts are dispensed simultaneously from the tube on the tooth brush applicator. An important aspect of the remineralization dentifrice of Winston and Usen is the addition of a divalent metal salt other than calcium, such as a magnesium salt, which at the slightly acidic pH, i.e., 5.0-6.5 of the toothpaste, retards the reaction between the calcium, phosphate, and fluoride salts so that sufficient free calcium and free fluoride are present to achieve effective remineralization. A very useful feature developed by Winston and Usen was the use of a partially water soluble calcium salt such as calcium sulfate as the active calcium remineralizing component. The use of the partially water soluble calcium salt ensured a sufficient concentration of calcium ions in solution to promote remineralization without compromising the free fluoride content of the aqueous composition. It was found that very highly water-soluble calcium salts, such as the chloride or nitrate salts, react readily with and overwhelm the free fluoride in the aqueous composition once mixed. Accordingly, the fluoride would rapidly precipitate as calcium fluoride from solution, thereby reducing the concentration of calcium and fluoride available for the remineralization process.

The use of bicarbonate salts as a dentifrice or the incorporation of such salts into dentifrice compositions is well known in the art of oral care. A renewed interest in incorporating bicarbonate salts into toothpaste has emerged in light of the success of the present assignee's Dental Care® and PeroxiCare® products. The addition of bicarbonate salts into dentifrices is beneficial for several reasons such as for providing good plaque removing capabilities as well as for improving the whitening properties of dentifrices. Importantly, bicarbonate salts provide a clean fresh feeling in the oral cavity after brushing and rinsing with water. It is also important to note that bicarbonate salts are very low in abrasion and therefore non-damaging to tooth enamel and dentin. Unfortunately, the mere addition of bicarbonate salts, such as an alkali metal or ammonium bicarbonate, into a dentifrice composition which further contains calcium would result in an undesirable precipitation of insoluble calcium carbonate and carbon dioxide gas would be evolved. If such a formulation was to be stored in a tube, the pressure of the gas could break open the tube. Whether for remineralization of sub-surface enamel lesions, or for providing the benefits of bicarbonate and/or fluoride to the teeth, premature reaction and precipitation of calcium with phosphates, fluorides, or bicarbonates adversely affects the usefulness of the oral care product.

U.S. Pat. Nos. 6,214,321 and 6,248,310 to Lee et al. are directed to oral care products containing a water soluble calcium phosphate salt or a monolithic combination of calcium and phosphate salts in a carrier with a first composition having a pH less than 7, and a second composition containing an alkaline material and a fluoride ion source in a carrier to achieve a pH greater than 7.5. The first and second compositions are separated from one another prior to use. When combined upon application to teeth, the first and second compositions generate hydroxyapatite depositing same on dental enamel. The alkaline materials are described as various alkali metal and alkaline earth metal bicarbonate, carbonate, oxides and hydroxide salts which can be present in the range of from 0.1 to 60%, preferably from 0.5 to 30% and more preferably from 1 to 20% by weight of the second composition. In the examples of the patent, when an alkali metal bicarbonate salt is used, levels of no more than 25% are disclosed. When an alkali metal carbonate salt is utilized, levels of 0.4% by weight or 6.5% by weight are specifically exemplified. The carbonate and bicarbonate salts are disclosed in the Lee et al. patents for the purposes of merely providing an alkaline material. No other purpose or advantage of these bicarbonate and carbonate salts is believed to be set forth.

Although the aforementioned references teach calcium-containing oral care products and the need for keeping calcium ions from prematurely reacting with phosphate and/or fluoride ions present as effective agents in the oral care products, it would be desirable to provide novel oral care products which contain bicarbonate and effectively release calcium, as well as fluoride ions, in the oral cavity as active agents.

### SUMMARY OF THE INVENTION

The present invention is directed to an oral composition capable of mineralizing the surface of dental enamel and which has improved stability to inhibit premature reaction between the active components of the composition. In particular, the composition is composed of:
(a) a first discrete cationic part containing an effective amount of at least one partially water soluble calcium salt;
(b) a second discrete anionic part containing a water soluble fluoride salt, at least one alkali metal or ammonium bicarbonate, and, optionally, a water soluble orthophosphate salt, wherein the first and second parts each have a pH in water such that a mixed aqueous solution of (a) and (b) has a pH of between about 7.0 and 10, and;
(c) a pharmaceutically acceptable carrier component;
   wherein the composition is adapted to simultaneously release the first and second parts when the composition is mixed with water and/or saliva to form the mixed aqueous solution.

The two-part oral composition of the present invention provides an effective amount of fluoride as an anti-caries agent, an amount of at least one bicarbonate salt so as to provide the advantages of bicarbonate as previously expressed with regard to oral care, and calcium to mineralize the surface of dental enamel.

A carbonate salt stabilizer may also be provided in the composition in amounts to help inhibit the immediate reaction of calcium with fluoride and, thus, reduce the precipitation of calcium fluoride. Excessive levels of the carbonate salt are avoided so as to inhibit the formation of insoluble calcium carbonate and prevent the loss of calcium ions from solution.

### BRIEF DESCRIPTION OF THE DRAWINGS

FIG. 1 is an SEM of the surface of an etched enamel specimen.

FIG. 2 is an SEM of the same enamel surface of FIG. 1 after treatment with the oral care composition of this invention.

### DETAILED DESCRIPTION OF THE INVENTION

The present invention is directed to a mineralizing two-part oral composition.

As used herein, the term “oral composition” means a composition which in the ordinary course of usage is not intentionally swallowed for purposes of systemic administration of particular therapeutic agents, but is rather retained in the oral cavity for a time sufficient to contact substantially all of the dental surfaces and/or oral tissues for purposes of oral activity. Non-limiting examples of oral compositions within the scope of this invention include toothpastes, mouth rinses, dental gels, professional treatment gels, tooth powders, and the like.

The two-part oral composition of this invention is composed of first and second discrete parts.

As used herein, the term “discrete” with respect to the two parts of the composition means that the parts do not react with one another prior to use of the composition. For example, as will be discussed in greater detail later herein, the two parts may be disposed in two separate compartments of a container or both parts may be disposed in a single medium but still remain unreacted with one another.

The first part of the oral composition of this invention contains an effective amount of at least one partially water soluble calcium salt, and the second part contains an effective amount of a water soluble fluoride salt and at least one bicarbonate salt. Optionally, the second part may further contain an effective amount of a water-soluble orthophosphate salt. Although possible, it is not preferable to include an orthophosphate salt in the first discrete part of the composition unless such orthophosphate salt is in the form of an insoluble calcium phosphate which cannot react and insolubilize the partially soluble calcium salt. Insoluble calcium phosphates might be utilized in the first discrete part of the formulation as abrasives to increase cleaning and stain removal. The addition of a carbonate salt to the second discrete part may be provided as an additional stabilizing agent to help inhibit premature reaction between the cationic calcium ion and anionic fluoride and orthophosphate ions.

As used herein, the term “effective amount” with respect to the amounts of the calcium, orthophosphate, and fluoride salt refers to those amounts of these salts that are sufficient to provide the desired benefit (i.e., anti-caries benefit and/or mineralization of surface enamel) while being safe to the hard and soft tissues of the oral cavity. It has been found that the surface imperfections in tooth enamel can be repaired and filled using compositions of this invention. The active ingredients in these compositions can react when applied to the tooth surface and form fresh mineral on etched or roughened enamel surfaces or within scratches and cracks at the enamel surface. The surface imperfections become filled in, the enamel is smoothed, and the teeth become glossier and healthier looking. A further significant advantage of the invention is that the smoothed enamel surfaces, due to treatment with products of the invention, have a reduced tendency to pick up stain. As will be shown in the examples that follow the description of this invention, the oral care composition of this invention can provide a layer of mineral continuous with the enamel surface to repair or otherwise finish damaged or roughened areas on the enamel surface. New mineral layers approaching 1 to 2 microns can be achieved by contacting the compositions of this invention with the teeth such as by brushing. This new and continuous mineral layer is not readily scraped off the tooth surface and is orders of magnitude thinner than, for example, tartar, which is often deposited as layers on enamel surfaces in thicknesses measured in millimeters. Frequently the thin layer, formed due to treatment with products of the invention, is harder than the underlying treated surface.

With respect to the orthophosphate and fluoride salts, the term “water soluble” means that at least 0.25 grams of the salt will dissolve in 100 milliliters of water at a temperature of 25° C. and a pH of 7.0.

As used herein, the term “partially water soluble” with respect to the calcium salt component refers to any toxicologically harmless calcium salt having a solubility in water which is greater than that of dicalcium phosphate dihydrate in an aqueous solution having a pH of about 7.0 and a temperature of about 25° C. but which is less than that solubility which would release more than about 1,400 ppm of calcium cations in such aqueous solution. In an aqueous solution having a pH of about 7.0 at a temperature of about 25° C., dicalcium phosphate dihydrate generally releases about 40 ppm of calcium cations. Thus, the partially water soluble calcium salt(s) used in the present invention generally has a solubility in water such that the salt releases more than about 40 ppm but no more than about 1,400 ppm of calcium cations in an aqueous solution having a pH of about 7.0 at a temperature of about 25° C. Preferably, the calcium salt(s) used in this invention has a solubility in water such that the salt(s) releases from about 100 ppm to no more than about 1,400 ppm of calcium cations in such aqueous solution. It should be noted, that the presence of other ionic species in solution, e.g. sodium and chloride ions, can somewhat increase the solubility of the partially soluble calcium salts in aqueous without reducing their usefulness in products of this invention.

Suitable partially water-soluble calcium salts include calcium sulfate, anhydrous calcium sulfate, calcium sulfate hemihydrate, calcium sulfate dihydrate, calcium malate, calcium tartrate, calcium malonate, calcium succinate, and mixtures of the foregoing. Anhydrous or hydrated calcium sulfate are preferred calcium salts.

The amount of the calcium salt in the oral composition of this invention preferably ranges from about 0.05% to about 10.0%. Amounts of the calcium salt from about 0.5% to about 5.0% by weight of the combined weight of the first and second parts are also exemplified.

A problem can result from the interaction of high concentrations of dissolved calcium ions with high concentrations of fluoride, bicarbonate, and/or phosphate ions when these components are brought together in use. Undesirably, this can result in rapid and premature precipitation of the end products as inactive suspended particles, rather than as new mineral formed on the surface of the treated tooth enamel. This invention overcomes this problem by providing the calcium component as a partially soluble salt. As a result, when the two discrete parts of the composition are mixed in aqueous media on teeth, the precipitation of the anionic and cationic components desirably occurs more slowly and in a more controlled fashion, and mineral forms on the surface of the tooth enamel and within the surface imperfections on the teeth.

The second part of the oral composition of this invention contains a water-soluble fluoride salt. Suitable water soluble fluoride salts for use in the present invention include inorganic fluoride salts such as soluble alkali metal or alkaline earth metal salts, for example, sodium fluoride, potassium fluoride, stannous fluoride, indium fluoride, zirconium fluoride, copper fluoride, sodium monofluorophosphate, ammonium monofluorophosphate, sodium fluosilicate, ammonium fluosilicate, and sodium fluozirconate. Organic fluorides, such as known amine fluorides, are also suitable for use in the present invention. Particularly useful water-soluble fluoride salts for use in the oral composition of this invention are sodium fluoride, potassium, stannous fluoride, sodium, or potassium monofluorophosphate.

When used in the oral composition of the present invention, the fluoride salt preferably constitutes from about 0.01% to about 5.0%, more preferably from about 0.02% to about 2.0%, by weight of the combined weight of the first and second parts. In general, concentrations of fluoride salts are such as to supply from about 650 to 2,000 ppm fluoride ion.

The second part of the oral composition of this invention further contains a water-soluble bicarbonate salt. The addition of bicarbonate to an oral product such as a toothpaste is advantageous in ways which have previously been described. Low abrasivity and efficacy in cleaning and stain removal, coupled with the ability to provide a fresh, clean feeling in the oral cavity, are some of the most useful advantages of adding a bicarbonate salt to the oral composition of this invention. Levels of bicarbonate salts in amounts of at least 2.0 wt. %, preferably at least 20 wt. % and, most preferably, at least 40 wt. % based on the total weight of the second part can be used. Generally, levels of bicarbonate salt(s) will range from 25-70 wt. %, with levels of 50-60 wt. % most preferred. Preferred water-soluble bicarbonate salts for use in the present invention include sodium bicarbonate, potassium bicarbonate, ammonium bicarbonate, and mixtures of the foregoing.

The second part of the oral composition optionally includes a water-soluble carbonate salt. Sodium carbonate, potassium carbonate, or a mixture of the foregoing can be used.

An effective amount of the water soluble carbonate salt if used in the oral composition of this invention can be added if needed to help stabilize the composition by inhibiting reaction between the calcium salt and fluoride salt. Most toothpastes in the United States contain about 1100 ppm fluoride ion. In the countries of Europe, the fluoride content is often higher. It is important for anti-caries protection that a sufficient amount of the fluoride material remain in the form of free fluoride in salivary aqueous solution in the oral cavity. Accordingly, the carbonate salt can be present in the second discrete part of the composition in amounts ranging from about 0.5 to about 4%, more preferably from about 1.0% to about 3.0%, by weight based on the weight of the second part. Excessive levels of carbonate salt are to be avoided to inhibit formation of a calcium carbonate precipitate. In the presence of bicarbonate salts, the carbonate salt can to be present in an amount sufficient to prevent the bicarbonate from reacting with the calcium to form carbon dioxide. When the calcium salt is partially water soluble (as defined hereinabove), in order to aid in preventing the calcium/bicarbonate salt reaction, the molar ratio of carbonate salt to calcium salt can range from about 0.1:1 to about 1:1, more preferably from about 0.2:1 to about 0.8:1.

The second part of the oral composition of this invention may further contain an effective amount of at least one water-soluble orthophosphate salt. Non-limiting examples of suitable water soluble orthophosphate salts include the alkali metal and ammonium salts of orthophosphoric acid, such as potassium, sodium, or ammonium orthophosphate; monopotassium phosphate; dipotassium phosphate; tripotassium phosphate; monosodium phosphate; disodium phosphate; trisodium phosphate; monoammonium phosphate; diammonium phosphate and triammonium phosphate.

Although not preferred, the water-soluble orthophosphate salt may be included in the first part of the oral composition of this invention. To prevent reaction between the calcium and phosphate salts and consequent formation of a calcium phosphate precipitate, the pH of the first part must be acidic. Thus, in this embodiment, the pH of the first part must be at most 6.0, preferably below 5.0, and most preferred at or below 4.5. Unfortunately, at a very low pH a sour taste may be imparted to the oral product requiring the need for increased levels of flavorants to mask the undesirable taste. It is preferred that the first part of the oral product be free of orthophosphate salts unless such salts are in the form of insoluble calcium phosphates which cannot react and insolubilize the partially soluble calcium salt.

Thus, calcium phosphate salts such as anhydrous dicalcium phosphate, dicalcium phosphate dihydrate or tricalcium phosphate can be included in the first part of the invention. These salts can usefully provide some abrasivity to the formulation to enhance cleaning but will not react with the partially soluble calcium salt and thereby inactivate it.

When used in the oral composition of the present invention, the water-soluble orthophosphate salt preferably constitutes from about 0.1% to about 15.0%, more preferably from about 0.5% to about 5.0%, most preferably from about 0.5% to about 2.0%, by weight of the combined weight of the first and second parts.

The first and second parts of the oral composition of this invention each have a pH value such that when the first and second parts are mixed with water and/or saliva to form a mixed aqueous solution, the solution will have a pH of between about 7 and 10. It has been found that at pH values below 7.0 there is insufficient stabilization of the fluoride and calcium active agents and, as such, premature reaction between the active agents can take place. At a pH above 10.0, the oral product would be irritating to the oral mucosa and would have an unpleasant taste.

The oral composition of this invention further contains a pharmaceutically acceptable carrier component. As used herein, the term “carrier” means a suitable vehicle which is pharmaceutically acceptable and can be used to apply the present compositions in the oral cavity. The term “carrier component” as used in the present invention refers to a single carrier in which the first and second parts are both disposed or two carriers, wherein one part is disposed in a first carrier and the second part is disposed in a second carrier.

The carrier for the components of the oral composition of the present invention can be any vehicle suitable for use in the oral cavity. Such carriers include the usual components of toothpastes, mouth rinses, dental gels, professional treatment gels, chewing gums, lozenges, confectioneries, tooth powders, and the like.

Toothpaste embodiments of the oral composition of this invention preferably include thickening agents, humectants, water, and abrasive polishing materials.

In preparing toothpastes, it is generally necessary to add some thickening material to provide a desirable consistency. Preferred thickening agents for use herein include carboxyvinyl polymers, carrageenan, hydroxyethyl cellulose, and water-soluble salts of cellulose ethers such as sodium carboxylmethyl cellulose (sodium CMC) or hydroxyethyl cellulose. Natural gums such as gum karaya, xanthan gum, gum Arabic, and gum tragacanth can also be used. Colloidal magnesium aluminum silicate or finely divided silica can be used as part of the thickening agent to further improve texture. Toothpaste embodiments of the oral composition of this invention preferably contain from about 0.1% to about 15.0% by weight of a thickening agent.

The water used in toothpaste and other embodiments of the oral composition of this invention is preferably deionized and free of organic impurities. Toothpaste embodiments of the oral composition of this invention usually contain from about 5% to about 50%, preferably from about 10% to about 40%, water by weight of the oral composition. These amounts of water include the free water which is added plus that which is introduced with other materials such as sorbitol.

Preferably, toothpastes further contain a humectant to prevent the toothpaste from hardening upon exposure to air. Certain humectants can also impart a desirable sweetness to toothpaste compositions. Liquid dentifrice and mouthwashes can also contain a quantity of humectant. Suitable humectants include glycerin, sorbitol, xylitol, polyethylene glycols, propylene glycol, other edible polyhydric alcohols, and mixtures thereof. Mixtures of glycerin and sorbitol are particularly useful. When present, humectants are generally used in an amount ranging from about 10% to about 70% by weight of the total oral composition.

Toothpastes and liquid dentifrices may also contain, if desired, an abrasive or polishing material, in addition to the bicarbonate salt. The abrasive polishing material contemplated for use herein can be any material which does not excessively abrade tooth enamel or dentin. These include, for example, silicas including gels and precipitates, calcium carbonate, dicalcium orthophosphate dihydrate, anhydrous dicalcium phosphate, calcium pyrophosphate, tricalcium phosphate, calcium polymetaphosphate, insoluble sodium polymetaphosphate, potassium metaphosphate, magnesium orthophosphate, trimagnesium phosphate, hydrated alumina, aluminum silicate, zirconium silicate, and resinous abrasive materials such as particulate condensation products of urea and formaldehyde. Mixtures of abrasives may also be used.

Preferred abrasive materials which may be admixed with the sodium bicarbonate include hydrated silica, silica gel or colloidal silica, and complex amorphous alkali metal aluminosilicates. The preferred abrasive polishing materials for use in the oral composition of this invention generally have an average particle size of from about 0.1 to about 30 microns, preferably from about 5 to about 15 microns. The abrasive is generally present in toothpaste formulations of the present invention at a level of from about 5% to about 50%, preferably from about 15% to about 25% by weight of the total composition.

Organic surface-active agents can be used in the dentifrices of the present invention to achieve increased cleaning action and improve the detergent and foaming properties of the dentifrices. Organic surfactants which may be so utilized can be anionic, nonionic or ampholytic in nature.

Examples of suitable anionic surfactants are water-soluble salts of the higher alkyl sulfates, such as sodium lauryl sulfate or other suitable alkyl sulfates having 8 to 18 carbon atoms in the alkyl group, water-soluble salts of higher fatty acid monoglyceride monosulfates, such as the sodium salt of the monosulfated monoglyceride of hydrogenated coconut oil fatty acids, alkyl aryl sulfonates such as sodium dodecyl benzene sulfonate, higher alkyl sulfoacetates, higher fatty acid esters of 1,2-dihydroxy propane sulfonate, and the substantially saturated higher aliphatic acyl amides of lower aliphatic amino carboxylic acid compounds, such as those having 12 to 16 carbons in the fatty acid, alkyl or acyl radicals, and the like. Examples of the last mentioned amides are N-lauroyl sarcosinate, and the sodium, potassium, and ethanolamine salts of N-lauroyl, N-myristoyl, or N-palmitoyl sarcosinate which should be substantially free from soap or similar higher fatty acid materials.

Other suitable surface-active materials include nonionic agents such as condensates of sorbitan monostearate with ethylene oxide, condensates of ethylene oxide with propylene oxide or, condensates of propylene glycol (available under the trademark “Pluronics”). Other examples of water-soluble nonionic surfactants useful in the dentifrices of the present invention are the condensation products of ethylene oxide with various other compounds which are reactive therewith and have long hydrophobic chains (e.g. aliphatic chains of about 12 to 20 carbon atoms), which condensation products (“ethoxamers”) contain hydrophilic polyoxyethylene moieties, such as condensation products of poly(ethylene oxide) with fatty acids, fatty alcohols, fatty amides, or polyhydric alcohols (e.g., sorbitan monostearate).

The various surfactants may be utilized alone, or in admixture with one another. In toothpastes, the aggregate amount of the surfactant or surfactants used is preferably within the range of about 0.05% to about 5%, more preferably, from about 0.1% to about 1.0%, by weight.

Any suitable flavoring or sweetening agent can be incorporated in the oral composition of this invention. Examples of suitable flavoring agents include flavoring oils such as oils of spearmint, peppermint, wintergreen, sassafras, clove, sage, eucalyptus, marjoram, cinnamon, lemon, orange, and methyl salicylate. Suitable sweetening agents include lactose, dextrose, maltose, levulose, sorbitol, xylitol, perillartine, sodium saccharin, aspartame, and acesulfame. Sucrose could also be used but is undesirable due to its cariogenic nature. Suitably, flavoring and sweetening agents may together constitute from 0.01% to 5% by weight of the oral composition of this invention. Preferably, the amount of flavoring agents is above 0.3%, e.g. 0.8 to 1.2%.

The oral composition of this invention may further contain a colorant. Colorants useful in the present invention include both pigments and dyes.

Pigments which can be used herein include non-toxic, water soluble inorganic pigments such as titanium dioxide and chromium oxide greens, ultramarine blues and pinks, and ferric oxides as well as water soluble dye lakes prepared by extending calcium or aluminum salt of FD&C dyes on alumina such as FD&C Green #1 lake, FD&C Blue #2 lake, FD&C Red #30 lake, and FD&C Yellow #1 lake. The pigments generally have a particle size of from 5 to 1,000 microns, preferably from 250 to 500 microns. The oral composition of this invention can contain from 0.5% to 3% by weight of a pigment.

Dyes which can be used in the present invention are generally food color additives presently certified under the Food, Drug & Cosmetic Act for use in food and ingested drugs, including dyes such as FD&C Red #3 (sodium salt of tetraiodofluorescein), FD&C Yellow #5 (sodium salt of a 4-p-sulfophenyllaxo-B-naphthol-6-monosulfonate), FD&C Green #3 (disodium salt of 4-{[4-(N-ethyl-p-sulfobenzylamino)-phenyl]4-hydroxy-2-sulfoniumphenyl)-methylene}-[1-(N-ethyl-N-p-sulfobenzyl)-3,5-cyclohexadienimine], FD&C Blue #1 (disodium salt of dibenzyldiethyl-diaminotriphenylcarbinol trisulfonic acid anhydride), FD&C Blue #2 (sodium salt of disulfonic acid of indigotin) and mixtures thereof in various proportions.

The oral composition of this invention can contain from about 0.0005% to about 2% by weight of a dye.

Various other materials may be incorporated in the oral care products of this invention including whitening agents and preservatives and other constituents. These adjuvants are incorporated in amounts which do not substantially adversely affect the properties and characteristics desired, and are selected and used in effective amounts depending upon the particular adjuvant.

The oral composition of this invention may also be in the form of a transparent or translucent gel. This is accomplished by matching the refractive index of the water humectant system with the abrasives and inorganic thickeners, if used.

Transparent gel embodiments of the oral composition of this invention preferably contain at least one gel-forming agent. Such gel-forming agents usually include known thickeners such as the alkali salts of polyacrylic acid and dehydrated silicon dioxide gels of particle size of from about 2 to about 20 microns and specific surface area of from about 200 to about 900 square meters per gram.

Professional gels can be formulated similar to toothpastes except with higher fluoride concentrations. Since professional gels are not designed for cleaning but only for applying fluoride, abrasives and other cleaning agents need not be included in the professional gel formulations of the present invention.

In the oral composition of this invention, the first and second parts are kept separate from one another until the composition is to be used. Separation of the two parts may be achieved by various means.

For example, the first and second parts may be kept separate from each other by disposing the parts as separate layers in a multiplayer product, for example, a two-layer toothpaste, a two-layer gel, and the like. For example, separation of the first and second parts may be achieved by having one of the parts be disposed in an aqueous carrier and the other part disposed in a non-aqueous, hydrophilic carrier.

Examples of suitable hydrophilic, non-aqueous carriers which can be used in the aforementioned embodiment of the present invention include non-aqueous solvents such as ethyl alcohol, glycerine, and propylene glycol. A particularly preferred hydrophilic, non-aqueous carrier is a polyethylene oxide having a molecular weight of about 400 (also know under the designation “Carbowax 400”).

In another embodiment of a toothpaste embodiment of the oral composition of this invention, the first and second parts may be disposed as two discrete stripes intertwined with one another, wherein one stripe contains the first part and has one color, and the other stripe contains the second part and has a second color. One or both of the stripes is non-aqueous and hydrophilic. In addition, one stripe may have a paste-like consistency and the other stripe may have a gel-like consistency.

In another suitable embodiment of the oral composition of this invention, the composition is in the form of an emulsion or dispersion wherein the first and second parts are present in different phases.

Separation may also be achieved by disposing the two parts in a single carrier, wherein the single carrier is non-aqueous and hydrophilic and capable of simultaneously releasing the two parts upon contact with water.

Yet another way to separate the first and second part is use of two carriers, wherein the first carrier is composed of a material in which the second part is insoluble but the first part is soluble, or vice versa.

In a particularly useful embodiment, the first and second parts may also be separated by a physical barrier such as when the two parts are disposed in separate compartments of a two-compartment pump container, two-compartment tube or two-compartment aerosol can. In this embodiment, which constitutes articles of manufacture within the scope of the present invention, the two parts are kept in separate compartments of the container during storage but are dispensed simultaneously with one another from the container. More specifically, when in the form of liquids, pastes, or gels, the two-part oral composition of this invention may be packaged in a suitable two-compartment dispensing container in which the first and second parts are maintained physically separated and from which the separated first and second parts are dispensed simultaneously. The configuration of a two-compartment dispensing container is not critical to the oral composition of this invention. Thus, physical barriers such as flat, elongated, rigid, or flexible dividers; or concentric dividers, whether insertable or integral with the container body, can be used. The separate compartment scan be of equal or unequal volumes.

In still another embodiment, the active components and adjuvants can be provided as a dry mix or powder which can be reconstructed with water immediately before use or with saliva during use.

### EXAMPLES

### Example 1

The following is a two-part dentifrice composition within the scope of the present invention. This composition can be provided in a tube in which the first and second parts are separated by a physical divider.

Part A (Cationic)

| Raw Material | %W/W |
|---|---|
| Glycerin | 34.550 |
| Sodium carboxymethyl cellulose | 0.500 |
| Xanthan Gum | 0.300 |
| Methyl paraben | 0.050 |
| Propyl paraben | 0.050 |
| Sorbitol (70%) | 29.297 |
| Purified water | 10.000 |
| Calcium sulfate | 4.000 |
| Sodium sulfate | 3.000 |
| Silicone dioxide (Aerosil 200VS) | 2.000 |
| Hydrated silica abrasive | 14.000 |
| Sodium lauryl sulfate | 0.750 |
| Flavor, color, and sweetner | 1.503 |
| Total | 100.000 |

### Part B (Anionic)

| Raw Material | %W/W |
|---|---|
| Glycerin | 26.810 |
| Potassium phosphate dibasic anhydrous | 1.800 |
| Sodium carboxymethyl cellulose | 0.500 |
| Purified water | 11.400 |
| Sodium fluoride | 0.440 |
| Sodium carbonate anhydrous | 1.800 |
| Sodium bicarbonate | 54.000 |
| Sodium lauryl sulfate | 1.250 |
| Flavor, sweetner | 2.000 |
| Total | 100.000 |

In this example the two parts are dispensed from a two compartment tube in the ratio of 0.45 parts of Part A to 0.55 parts of Part B.

### Example 2

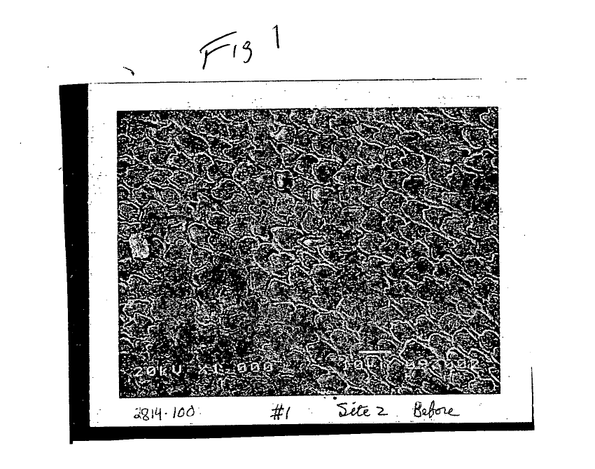

A sample of dental enamel having a size of a 3 ml diameter core was drilled from a tooth. The enamel sample was treated by contacting the specimen with an acid cola beverage for a time period of about one hour. The enamel specimen was rinsed with cold water. As can be seen from the SEM photograph of FIG. 1, the surface of the enamel has been substantially etched by the acid beverage. From FIG. 1 it can be seen that the horseshoe-shaped rods have been eroded to leave a pitted surface leaving the interstitial portions of the enamel protruding from the surface.

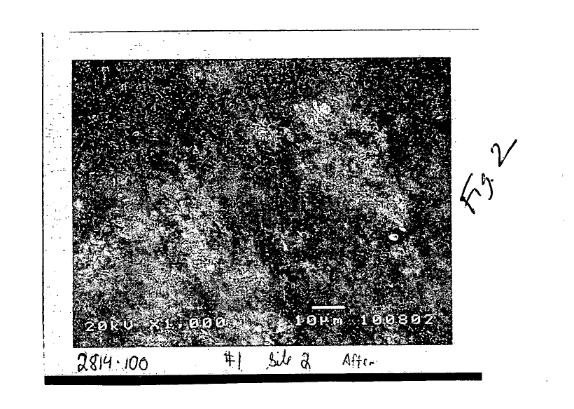

The etched enamel specimen was then contacted with a composition of the present invention containing a calcium sulfate salt, a fluoride salt, dipotassium phosphate and sodium bicarbonate in a carrier. The paste was diluted to yield a slurry containing one part paste to two parts water. The etched enamel specimen was soaked with the slurry for 5 minutes, rinsed with water, and soaked again. The specimen was soaked and rinsed 20 times. From FIG. 2, it can be readily seen that the enamel surface has been mineralized by contact with the composition of this invention. As shown in FIG. 2, the rods which were eroded have now been essentially filled in by the application of the composition of this invention. Approximately a 1 to 2 micron layer of new crystal structure was formed and resulted in smoothing out the roughened surface of the enamel.

1. A lateral current injection light emitting diode, comprising:
a first conductivity type semiconductor contact layer having at least one beveled sidewall;
a semiconductor active layer over a first surface of the first conductivity type semiconductor contact layer;
a second conductivity type semiconductor contact layer having a first surface over the active layer;
a transparent substrate below a second surface of the first conductivity type semiconductor contact layer;
a first electrode contacting the first surface of the first conductivity type semiconductor contact layer; and
a second electrode contacting a second surface of the second conductivity type semiconductor contact layer;
wherein a shape of a cross section of the transparent substrate is different from a shape of a cross section of the first conductivity type contact layer.

2. The light emitting diode of claim 1, wherein:
the first conductivity type semiconductor contact layer comprises an n-type III-V semiconductor contact layer containing Ga and N;
the active layer comprises at least one III-V semiconductor quantum well containing. In, Ga and N;
the second conductivity type semiconductor contact layer comprises a p-type III-V semiconductor contact layer containing Ga and N;
the first electrode comprises a metal electrode;
the second electrode comprises a reflective metal electrode contacting an entire second surface of the second conductivity type semiconductor contact layer.

3. The light emitting diode of claim 2, wherein:
the transparent substrate comprises a 125 to 200 micron sapphire substrate having at least one beveled sidewall;
the n-type contact layer comprises a 50 to 200 micron thick n-type GaN layer;
the p-type contact layer comprises a 0.05 to 10 micron thick p-type GaN layer;
the active layer comprises an InGaN quantum well between the p-type and the n-type GaN contact layers;
the first electrode comprises an Al layer and a Ti layer; and
the second electrode comprises a nickel oxide layer with or without Au dispersed in it, and an Al layer.

4. The light emitting diode of claim 2, wherein the at least one sidewall of the n-type contact layer is at a first angle such that light that is emitted from the active layer is incident on the at least one sidewall of the n-type contact layer at an angle greater than the critical angle.

5. The light emitting diode of claim 4, wherein:
the light incident on the at least one sidewall of the n-type contact layer is reflected from the at least one sidewall of the n-type contact layer through the transparent substrate at an angle less than the critical angle; and
the light incident on the reflective second electrode is reflected from the second electrode through the transparent substrate.

6. The light emitting diode of claim 2, wherein:
the n-type contact layer comprises a mesa having circular or oval cross section containing one sidewall; and
the second surface of the n-type contact layer is wider than the first surface of the n-type contact layer.

7. The light emitting diode of claim 2, wherein:
n-type contact layer comprises a mesa having polygonal cross section containing a plurality of sidewalls; and
the second surface of the n-type contact layer is wider than the first surface of the n-type contact layer.

8. The light emitting diode of claim 2, wherein a cross section of the transparent substrate comprises a polygonal cross section and a cross section of the n-type contact layer comprises an oval or a circular cross section.

9. The light emitting diode of claim 8, wherein:
the transparent substrate cross section has at least one non right angle, and contains a plurality of sidewalls; and
the n-type contact layer comprises a mesa having the circular or oval cross section containing one sidewall that is non contiguous with the plurality of the substrate sidewalls.

10. The light emitting diode of claim 2, wherein the transparent substrate comprises at least one beveled sidewall.

11. The light emitting diode of claim 1, wherein:
at least one substrate sidewall is not contiguous with the at least one corresponding beveled n-type contact layer sidewall; and
only a single light emitting diode is located on the substrate.

12. The light emitting diode of claim 2, wherein the first electrode comprises a reflective metal electrode substantially covering the at least one sidewall of the n-type contact layer.

13. The light emitting diode of claim 2, further comprising a package containing a first lead, a second lead and a light emitting surface, wherein:
the first electrode electrically contacts the first lead;
the second electrode electrically contacts the second lead; and
the transparent substrate is positioned toward the light emitting surface in a flip chip configuration.

14. The light emitting diode of claim 13, further comprising a plurality of light emitting diodes on the transparent substrate.

15. A lateral current injection light emitting diode, comprising:
a transparent substrate;
an n-type III-V semiconductor contact layer containing Ga and N, having a first surface containing a contact region and a second surface over the transparent substrate;
a III-V semiconductor quantum well active layer containing In, Ga and N over the first surface of the first conductivity type semiconductor contact layer;
a p-type III-V semiconductor contact layer containing Ga and N, having a first surface over the active layer;
a first metal electrode contacting the contact region of the n-type contact layer;
a second metal electrode contacting a second surface of the p-type contact layer; and
wherein at least one of the n-type contact layer and the substrate contain at least one beveled sidewall and at least one of the n-type contact layer and the transparent substrate contains a diamond or rhombus cross sectional shape with at least one non right angle.

16. The light emitting diode of claim 15, wherein:
the transparent substrate comprises a 125 to 200 micron sapphire substrate;
the n-type contact layer comprises a 50 to 200 micron thick n-type GaN layer;
the p-type contact layer comprises a 0.05 to 10 micron thick p-type GaN layer;
the active layer comprises at least one InGaN quantum well between the p-type and the n-type GaN contact layers;
the first electrode comprises an Al layer and a Ti layer; and
the second electrode comprises a reflective metal electrode comprising a nickel oxide layer with or without Au dispersed in it, and an Al layer, which substantially covers the second surface of the p-type GaN layer.

17. The light emitting diode of claim 16, wherein:
the at least one sidewall of the n-type contact layer and the at least one sidewall of the substrate are beveled;
the at least one sidewall of the n-type contact layer is at a first angle such that light that is emitted from the active layer is incident on the at least one sidewall of the n-type contact layer at an angle greater than the critical angle;
the light incident on the at least one sidewall of the n-type contact layer is reflected from the at least one sidewall of the n-type contact layer through the transparent substrate at an angle less than the critical angle; and
the light incident on the reflective second electrode is reflected from the second electrode through the transparent substrate.

18. The light emitting diode of claim 15, wherein:
the n-type contact layer comprises a mesa having the diamond or rhombus cross sectional shape with at least one non right angle and a plurality of sidewalls;
the second surface of the n-type contact layer is wider than the first surface of the n-type contact layer; and
the transparent substrate contains the diamond or rhombus cross sectional shape with at least one non right angle.

19. The light emitting diode of claim 15, wherein:
the transparent substrate contains the diamond or rhombus cross sectional shape with at least one non right angle.

20. The light emitting diode of claim 19, further comprising a package containing a first lead, a second lead, a light emitting surface and a phosphor, wherein:
the first electrode electrically contacts the first lead;
the second electrode electrically contacts the second lead;
the transparent substrate is positioned toward the light emitting surface in a flip chip configuration; and
the phosphor is located between the transparent substrate and the light emitting surface.

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. The light emitting diode of claim 15, wherein the n-type contact layer contains the diamond or rhombus cross sectional shape with at least one non right angle.

41. The light emitting diode of claim 15, wherein:
at least one substrate sidewall is not contiguous with at least one corresponding n-type contact layer sidewall; and
a single light emitting diode is located on the substrate.

42. A lateral current injection light emitting diode, comprising:
a first conductivity type semiconductor contact layer having at least one beveled sidewall;
a semiconductor active layer over a first surface of the first conductivity type semiconductor contact layer;
a second conductivity type semiconductor contact layer having a first surface over the active layer;
a transparent substrate below a second surface of the first conductivity type semiconductor contact layer;
a first electrode contacting the first surface of the first conductivity type semiconductor contact layer; and
a second electrode contacting a second surface of the second conductivity type semiconductor contact layer;
wherein:
at least one substrate sidewall is not contiguous with at least one corresponding first conductivity type contact layer sidewall; and
only a single light emitting diode is located on the substrate.

43. The light emitting diode of claim 42, wherein:
a shape of a cross section of the transparent substrate is different from a shape of a cross section of the first conductivity type contact layer; and
the light emitting diode semiconductor layers are deposited directly on the substrate by a semiconductor deposition method.

44. (canceled)

45. The light emitting diode of claim 42, wherein the first conductivity type semiconductor contact layer contains a diamond or rhombus cross sectional shape with at least one non right angle.

46. The light emitting diode of claim 1, wherein the first conductivity type semiconductor contact layer is deposited over the transparent substrate and the light emitting diode semiconductor layers are deposited directly on the substrate by a semiconductor deposition method.

47. The light emitting diode of claim 15, wherein the n-type III-V semiconductor contact layer is deposited over the transparent substrate and the light emitting diode semiconductor layers are deposited directly on the substrate by a semiconductor deposition method.

48. The light emitting diode of claim 1, wherein:
the transparent substrate comprises a first surface facing the first conductivity type semiconductor contact layer and a second flat surface opposite to the first surface; and
the light emitting diode comprises a completed light emitting diode.

49. The light emitting diode of claim 15, wherein the transparent substrate comprises a first surface facing the n-type III-V semiconductor contact layer and a second flat surface opposite to the first surface.

50. The light emitting diode of claim 42, wherein:
the transparent substrate comprises a first surface facing the first conductivity type semiconductor contact layer and a second flat surface opposite to the first surface; and
the light emitting diode comprises a completed light emitting diode.
